(19) United States
(12) Patent Application Publication
(10) Pub. No.: US 2025/0013919 A1
(43) Pub. Date: Jan. 9, 2025

(54) ALL-ELECTRONIC ANALYSIS OF BIOCHEMICAL SAMPLES

(71) Applicant: ProbiusDx, Inc., Fremont, CA (US)

(72) Inventor: Chaitanya Gupta, San Carlos, CA (US)

(21) Appl. No.: 18/577,033

(22) PCT Filed: Jul. 6, 2022
(86) PCT No.: PCT/US2022/036256

§ 371 Date: Jan. 5, 2024

Related U.S. Application Data
(60) Provisional application No. 63/219,338, filed on Jul. 7, 2021.

(51) Int. Cl. G06N 20/00

(52) U.S. Cl. CPC G06N 20/00

(57) ABSTRACT

A method includes (a) receiving data including current measurement data associated with a first sample by at least a sensor platform, metadata associated with the sensor platform, and an analysis to be performed on the current measurement data; (b) generating a feature set comprising coefficients by (i) selecting a set of basis functions from a plurality of predetermined learner functions indicative of properties of the electrochemical charge transfer, and (ii) generating the coefficients by projecting the current measurement data on the set of basis functions; (c) selecting a first Machine Learning (ML) model type from a predetermined set of ML model types, the selecting based on the received user-selected analysis; and (d) providing the feature set to an ML model characterizing by the selected ML model type, the first ML model configured to characterize the first sample.

### RELATED APPLICATION

This application claims priority to and benefit of U.S. Provisional Application No. 63/219,338, filed on Jul. 7, 2021; the contents of which are hereby incorporated in its entirety.

### FIELD

The present disclosure relates generally to devices and methods useful for detection and characterization of biochemical samples.

### BACKGROUND

Traditional methods of bioanalysis include preparation of a sample including a target analyte and analyzing the analytes using analyte-specific chemistries (e.g., detect the analyte by attaching to the analyte). The preparation of the sample can include stripping the biological matrix of the sample from the analyte to be detected to present a “clean” sample for detection. The detection can be performed by the sensor including a physical transducer that converts information about the presence of the analyte to a measurable signal (either via the intermediate binding step or directly as done in mass spectrometry). The interaction of the transducer with the to-be-detected analyte can require intermediate cleaning steps to ensure there is no interference in the transducer signal from other biological species in the stripped-down and sample-prepared matrix.

The traditional approach can require target-specific chemicals, biological reagents and cleaning steps to be incorporated as part of a multi-step protocol in the detection of analytes. The use of these target-specific chemicals, biological reagents and cleaning steps also necessitates a-priori hypothesis/knowledge of the target that will be detected as part of the workflow. Furthermore, each time a new analyte in the sample needs to be analyzed, the sample may need to be prepared again. As a result, traditional methods of bioanalysis can be cumbersome, inefficient and expensive.

Accordingly, there remains a need in the art for methods and devices for hypothesis-free interrogation of analytes in samples that does not require extensive preparation and use of analyte-specific chemistry. These methods can be used to characterize complex biological samples (e.g., via an electrochemical interface based on all-electronic detection), and analyze the characteristic data using machine learning algorithms.

### SUMMARY

This section provides a general summary of the disclosure, and is not comprehensive of its full scope or all of its feature.

In one aspect, provided herein are methods for characterizing biological samples, the method including (a) receiving data comprising current and voltage measurement data associated with a first sample by at least a sensor platform, metadata associated with the sensor platform, and a user-selected analysis to be performed on the current measurement data, wherein the current measurement data includes current measurement signal data as a function of voltage applied by the sensor platform on the first sample and a measurement time and voltage measurement data includes voltage measurement signal as function of applied set point voltage and a measurement time (b) generating a feature set comprising a plurality of coefficients by at least (i) selecting a set of basis functions from a plurality of predetermined learner functions indicative of properties of the electrochemical charge transfer at a sensor interface of the sensor platform, and (ii) generating the plurality of coefficients by at least projecting the current measurement data on the set of basis functions; (c) selecting a first Machine Learning (ML) model type from a predetermined set of ML model types, the selecting based on the received user-selected analysis; and (d) providing the feature set to an ML model characterizing by the selected ML model type, the first ML model configured to characterize the first sample.

In any of the embodiments above and herein, the metadata associated with the sensor platform includes physical properties of the sensor platform indicative of the electrochemical charge transfer at the sensor interface and/or operational properties of the sensor platform associated with detection of the current measurement signal.

In any of the embodiments above and herein, the received data further includes one or more of (a) data of the source of the first sample, (b) quantitative information associated with analyte species determined from other analysis methods; (c) date and time of first sample collection, storage and re-thaw; (d) one or more quality controls applied to the first sample during collection, storage; (e) any quality control applied to first sample just before analysis; (f) information about co-morbidities of first sample source; (g) disease-relevant phenotypes for first sample.

In any of the embodiments above and herein, selecting the set of basis functions includes selecting a first set of learner functions and a second set of learner functions from the plurality of predetermined learner functions; fitting the current measurement signal data with the first set of learner functions and the second set of learner function; and calculating a first prediction error and a second prediction error associated with the fitting of the current measurement signal with the first set of learner function and the second set of learner function, respectively.

In some implementations, the method further includes selecting one of the first set of learner functions and the second set of learner functions based on the first prediction error and the second prediction error. In some implementations, the method further includes selecting the first set of learner functions wherein the first prediction error is smaller than the second prediction error.

In any of the embodiments above and herein, the method further includes selecting a first ML model having the first ML model type, wherein the first trained ML model is characterized by the first model type; determining that the first ML model does not require further training; and generating an output by the first ML model configured to receive the feature set and user defined metadata as an input. In some implementations, the user specified analysis includes assigning a class to an analyte in the first sample and wherein the first ML model is a classifier configured to assign the class to the analyte. In some implementations, the user-specified analysis includes quantification of concentration of an analyte in the first sample.

In any of the embodiments above and herein, the method further includes selecting a second ML model having the first ML model type, wherein the first trained ML model is characterized by the first model type; determining that the second ML model requires further training; training, using a training model, the second ML model based on training data including one or more of first sample data, metadata associated with detection of current measurement signal and previously generated output of the second ML model; and generating an output by the second ML model configured to receive the feature set and user defined metadata as an input.

In some implementations, the method further includes training the second ML model to assign a class type associated with the first sample, wherein the second ML model is a classifier configured to assign the class to an analyte, wherein the training data is based on one or more samples assigned the class type, wherein training the classifier includes determining classifier boundary; and assigning the class type to the analyte in the first sample using the trained second ML to assign a class to the sample.

In some implementations, the method further includes defining calibration analyte samples; analyzing the calibration analyte samples; training the second ML algorithm based on a Scattered Component Analysis (SCA) to determine a projection vector that maximizes similarity to analyte-specific reference sample data while minimizing similarity to matrix-specific reference data and/or similarity to chemically and structurally similar analyte reference data, to digitally subtract the contribution of the background and other similar analytes to the signal; and determining a concentration of the analyte by at least projecting, by the trained second ML algorithm, the sample data onto the projection vector.

In any of the embodiments above and herein, the method further includes determining that an ML model having the first ML model type does not exist; identifying a second sample based on a predetermined relationship with the first sample; identifying a third ML model and second training data associated with the second sample, the second training data including one or more of the second sample data, metadata associated with detection of a current measurement signal associated with the second sample and previously generated output of the third ML model; training, using a training model, the third ML model based on the second training data; and generating an output by the third ML model configured to receive the feature set and user defined metadata as an input.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a schematic illustration of biological sample characterization;

FIG. 2 schematically illustrates an exemplary method for characterizing biological samples;

FIG. 3 illustrates and exemplary method of raw data measurement including current and voltage measurement data in the method described in FIG. 2;

FIG. 4 illustrates an exemplary method for generating a feature set in the method described in FIG. 2;

FIG. 5 illustrates an exemplary method for characterizing biological sample using machine learning algorithm in the method described in FIG. 2;

FIG. 6 illustrates an exemplary flow-chart for selecting a machine learning algorithm for the characterization of biological sample;

FIG. 7 illustrates an exemplary flow-chart for classifying a target phenotype in a sample;

FIG. 8 illustrates an exemplary flow-chart for quantifying a target analyte in a sample;

FIG. 9 illustrates an exemplary method for performing transfer learning;

FIG. 10 illustrates and exemplary decentralized deployment of machine learning (ML) or Artificial intelligence (AI) driven workflow;

FIG. 11 illustrates an exemplary method for biochemical phenotyping of disease biology in mouse whole blood, followed by a step-by-step characterization of how that phenotype is expressed in terms of relationships between different disease-relevant pathways where the characterization process involves quantitative estimation of biomarker concentrations as well as estimation of the correlations between the simultaneous expression of biomarkers in the same sample;

FIG. 12 illustrates an exemplary method for biochemical phenotyping of tuberculosis in human plasma samples; and

FIG. 13 illustrates an exemplary implementation of after-the-fact HIV classification on data used to identify the tuberculosis phenotype.

FIG. 14 illustrates an exemplary implementation of biochemical phenotyping of two isoforms of insulin (Humalog and Toujeo) in their pure forms, followed by a quantitative calibration curve for the measurement of Humalog in a batch of Toujeo and vice-versa.

FIG. 15 illustrates prediction accuracy for models developed for quantitative analysis of circulating liver enzymes ALT, AST and Albumin in rat serum. Types of samples used to develop the training models are listed below each figure as exemplars for the model training samples

### DETAILED DESCRIPTION OF THE DISCLOSURE

The present disclosure generally relates to, inter alia, methods for characterizing biological samples (e.g., electrochemical solution including analytes and redox species). The method for characterizing the biological sample can include a workflow that is universal (e.g., not specific to a give analyte due to analyte-specific chemistry) and simplified (e.g., does not require extensive sample preparation). In some implementations, the method relies on a biological sample measurement method (e.g., by a sensor platform including a consumable and an instrument) and machine-learning (ML) enabled data analysis stack, where the appropriate analysis can be customized from a suite of available ML models, to predict the sample phenotype or the quantitation of specific biological characteristics, including biomarkers with a high degree of sensitivity and specificity.

An assay is described as a process of assigning a phenotype class to a sample or assessing the expression/concentration of one or more analytes in a sample. In some implementations, the system (or sensor platform) for performing the assay can include three elements: the consumable, the instrument and one or more computing systems for executing feature-set extraction (e.g., from raw data acquired by consumable/instrument detection) and analysis software stack.

Each element of the system could have multiple implementations. Each implementation can be informed by customer workflows and the sample type being analyzed. For example, selection of a particular implementation can require assessment of trade-off between throughput, power, footprint and desired noise power-spectral-density (PSD) performance. In some implementations, the consumable and/or instrument can be modified to tailor to specific applications.

The consumable can include a sensor with an interface geometry configured to interface with the sample including the analyte. The interface geometry can include nanoscale electrochemical interface described in U.S. application Ser. No. 16/016,468 and U.S. Pat. No. 9,285,336 which have been incorporated herein by reference in their entirety. The consumable can be integrated with a sample collection mechanism (e.g., syringe, pipette, breath analyzer). Alternately, the consumables can be integrated with a sample storage device (e.g., storage cap, vial/test tube, vacutainer, beaker, dried spot card, microtiter plate, culture/other flask, ufluidic cartridge, etc.). In some implementations, the consumable and/or the instrument can be integrated with sample handling robots. The instrument can be integrated with the consumable (e.g., can be configured to receive an electric signal indicative of detection by the consumable). The instrument can have a low throughput (e.g., single consumable read), a medium throughput (e.g., 8 consumable read) or a high throughput (e.g., 24-1536 consumable read). The medium and high throughput instruments can perform multiple readouts/scan of samples in multiple consumables.

The computation of raw data acquired by the instrument (e.g., using a Machine Learning model) can be executed locally (e.g., local compute) or on a cloud (cloud compute). The determination of whether to perform the computation locally, on a cloud or a combination thereof can be based on internet connectivity, need to preserve data security and/or quick time to result.

In some implementations, each system element (e.g., consumable unit, instrument unit, differentiated data sampling and analysis method) can be identified using a unique identifier. A unique identifier documents the processes used to prepare the corresponding system element as well as the quality control it was subject to prior to release. The unique identifiers can characterize the specifications required of the system elements, and tolerances around said specifications. This can allow for transduction of vibrational mode information into electrochemical signals which can then be digitized, transmitted and analyzed through suitable computational and machine learning models.

In some implementations, work flow described herein can include pipetting a small volume (2-100 ul) of homogenized sample into the consumable element of the system. Each sample can be associated with labels that provide a meta description of the sample origin and/or its biochemistry, and the physical sample itself could have gone through processing (e.g. related to how it was stored and retrieved) and quality assessment prior to aliquoting into the consumable. The workflow itself does not require any sample preparation to enable the measurement.

In some implementations, the consumable can be mated with the instrument, either before or after manual or automated dispensing of the sample. In some implementations, an instrument interface can allow the user to enter and/or associate relevant sample metadata and trigger a measurement on the sample. The measurement process can include a set of automated checks to verify the consumable-to-instrument connection, followed by a scan of a voltage applied to an electrochemical sensor imbedded in the consumable element, across a desired range of values. Recordings of the time-dependent electrochemical current, voltage (raw data) are made available to the backend analysis stack. In some implementations, measurement logs of environmental sensors embedded within the instrument can generate readouts that assess the environment within which the measurement was made.

In some implementations, the raw data can be pushed through a feature extraction algorithm that converts the raw data into a set of features in a high dimensional signal space that can be analyzed further. The generated feature-set can be qualified by the operator/user-specified metadata and the metadata associated with the features can be dynamic and evolve with time (e.g. as more information is available about the sample, either via third party users or through further subsequent analysis). The additional information can be included as additional metadata applied to the existing feature-set.

Once the set of features (or feature set) is extracted from the data, the obtained feature-set is subject to an analysis process.

In some implementations, the feature set can be included as part of a larger/layered training dataset that can be already available from databases of available feature-sets. For example, an aggregated training dataset including the user-assigned metadata, labeled training sample feature-sets drawn from other databases (e.g., associated with a different sample(s)), etc., can be used to provide a classification domain of the incoming feature-set. The aggregated training dataset can be used to train, validate and calibrate machine learning models for assaying the sample.

In some implementations, the feature set can be added to a database of metadata labeled feature-sets, where the training dataset can be dynamically aggregated with the addition of more feature sets extracted from additional sample measurements (e.g. using the consumable and instrument described above). The samples used to generate the training dataset can be chosen to sensitize a classifier to elements of the feature-set that are specific to the target (phenotype or analyte) that need to be detected (e.g., a biomarker known to be associated with the biology in the sample). The aggregated training dataset can be used to train, validate and calibrate machine learning models for assaying the sample.

In some implementations, the feature sent can be added to a database of metadata labeled feature sets, where the training dataset can be dynamically aggregated with the addition of new feature sets. The new feature dataset can be determined from a deterministic mathematical simulation of electrochemical charge transfer in the presence of elevated intensities of specific target or from a predictive estimation using artificial intelligence constructs like neural networks or deep learning networks that characterize expected feature-set values for given target from known feature-set distributions of closely related phenotypes or analytes.

In some implementations, the feature sent can be added to a database of metadata-labeled and transformed feature sets obtained from previously measured, similar sample types (e.g. similar biological matrices across specie types like rat and human serum), where the feature-set transformation is applied to mathematically project the similar sample domain onto the domain of the sample on which a current assay is being performed. The thus-aggregated training dataset can be used to train, validate and calibrate machine learning models for assaying the sample to determine the presence and concentration of a particular analyte or to phenotype the sample (e.g. sample has a specific diabetes phenotype).

In some implementations, the feature sent can be used as a blind sample on which the assay is performed (e.g., with an available, trained, validated and calibrated machine learning model that can be selected from a menu of available models). The output from analysis of the blind sample can become metadata that qualifies the feature-set associated with the ‘blind’ sample, which then allows the blind sample feature-set to be used as training data in another assay.

In some implementations, the feature analysis can include a statistical comparison of an unknown or blind sample feature-set against a set of ‘known’ or ‘reference’ features that are derived from well-characterized training samples. The known or reference training features can include metadata labels that apriori describe the state of the target in the sample. For example, the metadata labels can include the expected variability of the target-specific features due to the variability in the biological matrix in which the target exists. The references can represent a ground truth baseline associated with the target with respect to which the assay is being performed and this ground truth may be arrived at using real-world samples or ‘contrived’/artificially generated samples, as produced by methods described herein. The known or reference training features can be generated using methods and devices described herein and converted into a set of equivalent labeled features. The statistical comparison to the references can include a mathematical transformation of the blind sample feature-set onto a domain defined by the reference features, after digital removal/subtraction of the feature components from the sample matrix, which can results in a reference-specific digital filter with which the sample features get analyzed for the assay procedure.

In some implementations, the generated feature-set and accompanying metadata labels can become a virtual representation of the sample that can be archived in a digital database for posterity and used repeatedly for forensic analysis of the sample with the different biological hypothesis that leads to a new training dataset and new machine learning models for analysis, as part of an aggregated training dataset for a new assay, etc.

In some implementations, the input of feature generation (or feature extraction) can include measurement data (e.g., raw electrochemical measurement data generated based on detection by the instrument via the consumable). The measurement data can include current or voltage measurement as functions of time. The input of the feature extraction can include sample metadata, measurement logs, consumable and instrument identifiers, etc. In some implementations, the feature extraction can include ensuring that the measurement data has a desirable form (e.g., suitable for extraction of feature set). The output of the feature generation can include a feature set matrix.

In some implementations, the input of the biological sample characterization (or feature analysis) can include the feature set matrix (e.g., generated by feature generation) and associated metadata. The metadata can be associated with measured sample that can be measured against existing model or that can be added to a reference database. The reference can include aggregated from existing databases, aggregated and transformed from existing databases, developed and aggregated from one or more of (a) new real world samples relevant to the biochemical phenotype; (b) new contrived/artificially prepared physical samples in which a target or target-surrogate is inoculated within controlled matrices (blank, sample-specific) to generate feature-sets corresponding to increased target presence in the sample; and (c) contrived ‘digital’ samples where the reference feature-sets are generatively determined from output of mathematical simulation or predictive estimation using artificial intelligence constructs like neural networks or deep learning networks. The output of feature analysis can include phenotype class determination, concentration/expression of analyte, etc.

Some implementations of the method described herein can enable comprehensive biochemistry snapshots, hypothesis-free analysis of digital twins, longitudinal personalized baselines, epidemiological (population wide) health characterization, enabling efficient feedback loops with inputs from health professionals and the marketplace.

In some implementations, a broad spectrum of vibration information can be extracted (e.g., indicative of vibrational properties of analytes and redox species in the sample) and a digital signature can be generated. The digital signatures can be used (e.g., mined) for target specie expression. In some implementations, the methods described herein do not require a chemical label, a probe or purifying the sample and are agnostic to the type of analyte being assayed. In some implementations, the methods described herein can enable the study of the consequences of phenotype, gene expression, environmental factors and pharmacology in an integrated manner within a biological matrix.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Methods of the Disclosure

As outlined above, some aspects of the disclosure provide methods for characterizing biochemical sample.

In some implementations, determination of a state of the target (e.g., class or phenotype, analyte concentration, analyte expression, etc.) can enable hypothesis-free interrogation of samples generated from biochemical experiments. For example, a) a sample phenotype expressed as a set of digital features that can allow for the capture of characteristic data (e.g., current measurement data, voltage measurement data associated with a sample) that can include broad-spectrum biochemistry of the sample (e.g., as a compact digital file). In some implementations, a feature set (e.g., including a plurality of coefficients) can be extracted from the captured characteristic data. The feature set can encode, for example, the expression of a disease, applied therapeutic intervention within the sample. In some implementations, this expression-rich feature-set can subsequently be compared against a suite of available references to determine the quantitative expression of multiple analytes in the sample which could define a novel biomarker profile for investigations into disease diagnostics and treatments as well as to understand how different therapeutic modalities impact disease (and healthy) biology. The biomarker profile can span multiple length scales from small molecules to single cells. For example, the biomarker profile can include of panels of several co-expressed biomolecular species in the sample.

The feature sets associated with the sample can be queried using digital filters (e.g., digital filters defined by machine learning models developed from references). Querying feature sets can obviate the need for physical sample preparation before measurement (e.g., since the matrix contribution can be removed digitally from the signal feature-space) and also enable the multiplexed analysis of the sample dataset without necessitating extra physical sample to be drawn for the analysis. In some implementations, the assay or the sample analysis can be virtualized into a software environment via a series of mathematical transforms on the feature-sets generated from the sample data. For example, the analysis can be customized and modified by the manipulation of the underlying mathematical algorithms. The physical use of chemicals, biological reagents, probes and labels as well the complex workflows and instruments associated with sample preparation in traditional life-sciences research tools can be replaced with a single instrument type, consumable type and suite of mathematical functional transforms.

The target- and matrix-agnostic nature of the system and the scalability of the electrochemical transduction method can allow for broad applicability in disease research, determination of therapeutic efficacy and toxicity, diagnostic screening/triaging and in industrial quality control. In some implementations, the methods described above can be used to a) log sample phenotypes as high dimensional feature-sets and/or b) measure the intensity of specific targets in the sample by leveraging a combination of digital matrix subtraction and digital filter engineering from the reference or training data.

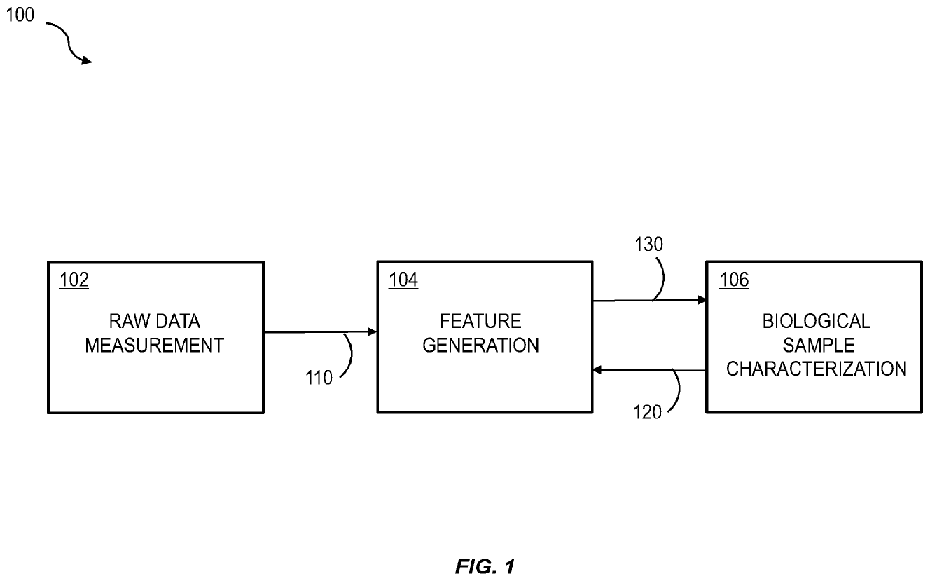

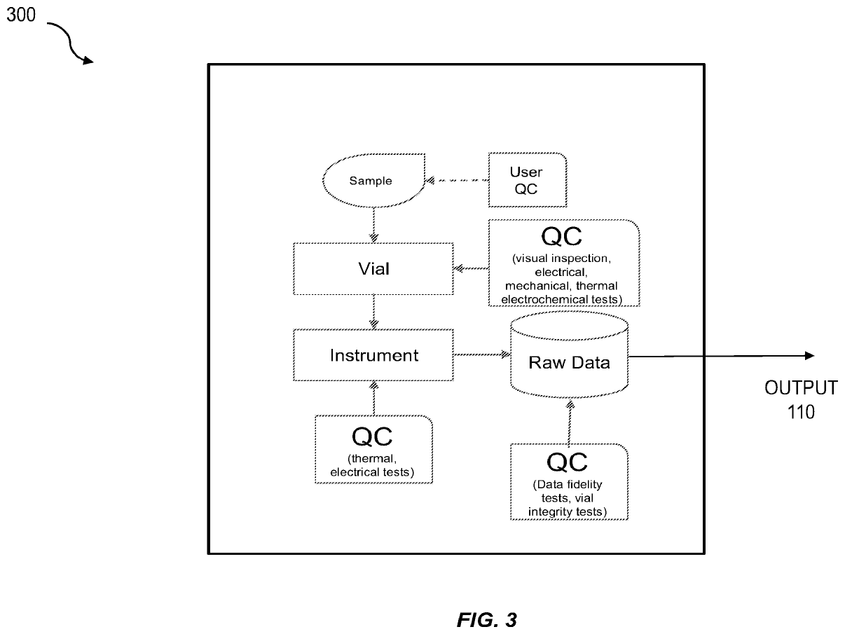

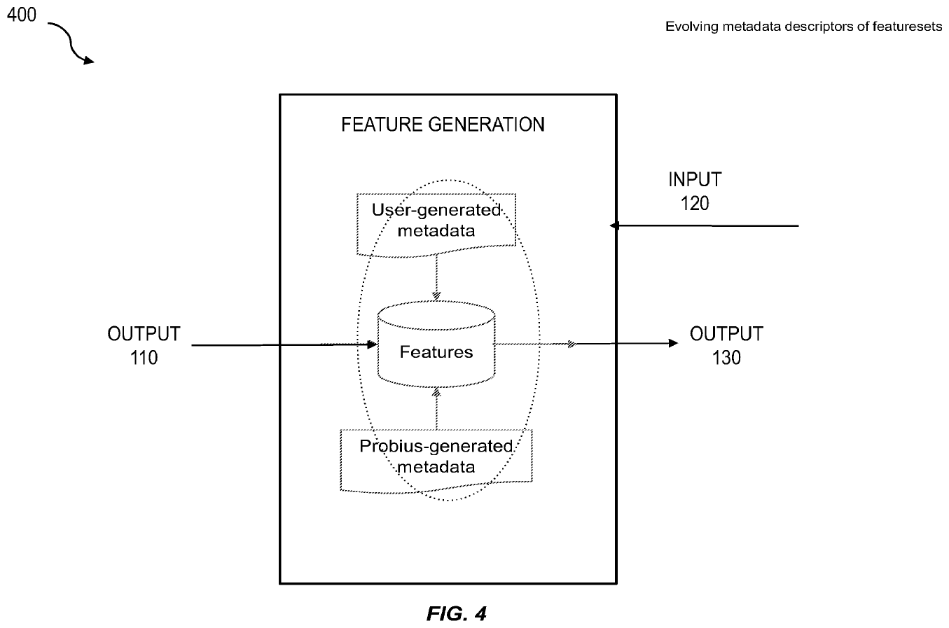

FIG. 1 is a schematic illustration of biological sample characterization process 100. The characterization process 100 can include raw data measurement 102, feature generation 104 and biological sample characterization 106. The raw data measurement 102 can include performing a voltage scan of the sample (e.g., by applying multiple voltages across the sample) and detecting the resulting current signal including time-dependent electrochemical current (e.g., via the sensor interface in the consumable). The current signal can be detected by the instrument (e.g., including a high-gain, low noise feedback circuit). The high-gain, low noise feedback circuit is described in U.S. application Ser. No. 16/096,893 which has been incorporated herein by reference in its entirety. The raw data measurement process is further described in FIG. 3 below. The output 110 of the raw data measurement can be provided for feature generation 104. The output 110 can include raw electrochemical measurement data (e.g., current, voltage measurement as a function of time, sample metadata, measurement logs, consumable and instrument identifiers, etc. The feature generation 104 can receive the output 110 as an input. The feature generation 104 can also receive input 120 including metadata from the biological sample characterization 106. The feature generation 104 and generate a feature set. The feature set and the associated metadata can be provided to the biological sample characterization 106 via the output 130. The feature set generation process is further described in FIG. 4 below. Based on the output 130, the biological sample characterization 106 can classify an analyte/quantify the concentration of the analyte in the sample associated with raw data measurement 102.

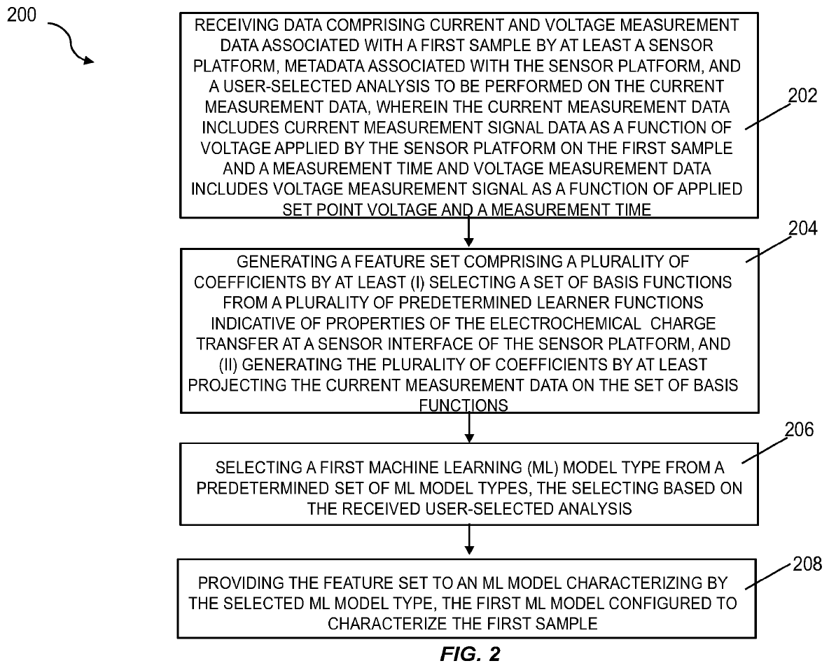

FIG. 2 schematically illustrates an exemplary method 200 for characterizing a biological sample. At step 202, data including current and/or voltage measurement data (e.g., raw measurement data) associated with a first sample (e.g., detected by at least a sensor platform including a consumable and an instrument), metadata associated with the sensor platform, and a user-selected analysis to be performed on the current measurement data is received. The current measurement data includes current measurement signal data as a function of voltage applied by the sensor platform on the first sample and a measurement time and/or voltage measurement data includes voltage measurement signal as function of applied set point voltage and a measurement time.

FIG. 3 illustrates an exemplary method of raw data measurement. A sample (including an analyte) to be analyzed can be placed in a sensor platform including a vial and an instrument. The sensor platform can include all-electronic high-throughput analyte detection system described in U.S. application Ser. No. 16/194,208 which has been incorporated herein by reference in its entirety. A small volume of suspended sample can be aliquoted into the vial. The sample may not need to be pre-prepared (e.g. by stripping biological matrix of the sample). As discussed later, a ML model can be used to remove signature of background matrix from sample feature dataset (digital sample preparation). In some implementations, a sample preparation protocol can be added to the analysis methodology on right (e.g., sample could be physically prepared with a specific protocol prior to pipetting into vial). In some implementations, the sensor platform can modified with specific scan parameters to tailor analysis to specific application. In some implementations, each step in physical analysis can be quality controlled (QC), with a set of associated tests.

The metadata associated with the sensor platform can include physical properties of the sensor platform indicative of the electrochemical charge transfer at the sensor interface (e.g., pore size, noise PSD, etc.) and/or operational properties of the sensor platform associated with detection of the current measurement signal. (e.g., manufacturing run number, date, leak test etc.). In some implementations, the data received at step 202 can further includes one or more of (a) data of the source of the first sample (e.g., individual's age and/or health conditions from whom the analyte is obtained, source animal's specie, etc.), (b) quantitative information associated with analyte species determined from other analysis methods; (c) date and time of first sample collection, storage and re-thaw; (d) one or more quality controls applied to the first sample during collection, storage; (e) any quality control applied to first sample just before analysis; (f) information about co-morbidities of first sample source; (g) disease-relevant phenotype for first sample (e.g., determined using the analyte classification method described herein). The user can select the analysis to be performed on the sample provided in the vial (e.g., via a graphical user interface display space in the sensor platform).

Returning to FIG. 2, at step 204, a feature set comprising a plurality of coefficients can be generated. In some implementations, the feature set generation can include selecting a set of basis functions from a plurality of predetermined learner functions indicative of properties of the electrochemical charge transfer at a sensor interface of the sensor platform. Feature set generation can further include generating the plurality of coefficients by at least projecting the current measurement data on the set of basis functions. The basis functions can be indicative of a probability of electronic transition from metal to redox species in the sample (or vice versa), given a vibrational mode of frequency ω mediating the transition.

The current measurement for a given voltage “V” can be represented as:

$$I ( V , t ⊂ [ 0 , Δ {t}_{sample} ] ) = {\sum }_{H} {a}_{H} A ( \{ {p}_{H} \} )$$

The above equation represents, ensemble decomposition of current I using parametric basis function A (parameterized by pₙ). Unlike standard transforms (e.g., fourier transform), the parametric basis function A can depend on properties of the consumable, instrument (e.g., sensor-sample interface) and the physics of charge transfer process at the interface.

In some implementations, selecting the set of basis functions can include selecting a first set of learner functions and a second set of learner functions from the plurality of predetermined learner functions. This can be done, for example, via an ensemble generator, where a coarse regularized optimization selects those members of the plurality of learners that are best fit to the current voltage profile of the electrochemical system. The selecting of the set of basis function can also include fitting the current measurement signal data with the first set of learner functions and the second set of learner function. For example, a fit of ensemble representation can be optimized by minimizing bias-variance tradeoff (e.g., trade-off in the estimates of n, an and pₙ). Furthermore, a first prediction error and a second prediction error associated with the fitting of the current measurement signal with the first set of learner function and the second set of learner function, respectively, can be calculated. Based on the first prediction error and the second prediction one of the first set of learner function and the second set of learner function can be selected. For example, the set of learner function with the smaller prediction error can be selected.

Returning back to FIG. 2, at step 206, a first Machine Learning (ML) model type can be selected from a predetermined set of ML model types. The selecting based on the received user-selected analysis. For example, the ML model type can include a classifier (e.g., for assigning a class to an analyte) or a quantifier (e.g., determine the concentration of an analyte in the sample). In some implementations, an ML model can be selected based on the model type (e.g., included in or determined from the received user-selected analysis). In some implementations, it can be determined that the selected model does not require further training, and the ML model can used to generate an output based on the feature set and user defined metadata. In some implementations, the user specified analysis can include assigning a class to an analyte in the first sample. The selected first ML model can be a classifier configured to assign the class to the analyte (e.g., the output of the first ML model can include the classification of the analyte). In some implementations, the user-specified analysis includes quantification of concentration of an analyte in the first sample (e.g., the output can include concentration of analyte in the sample).

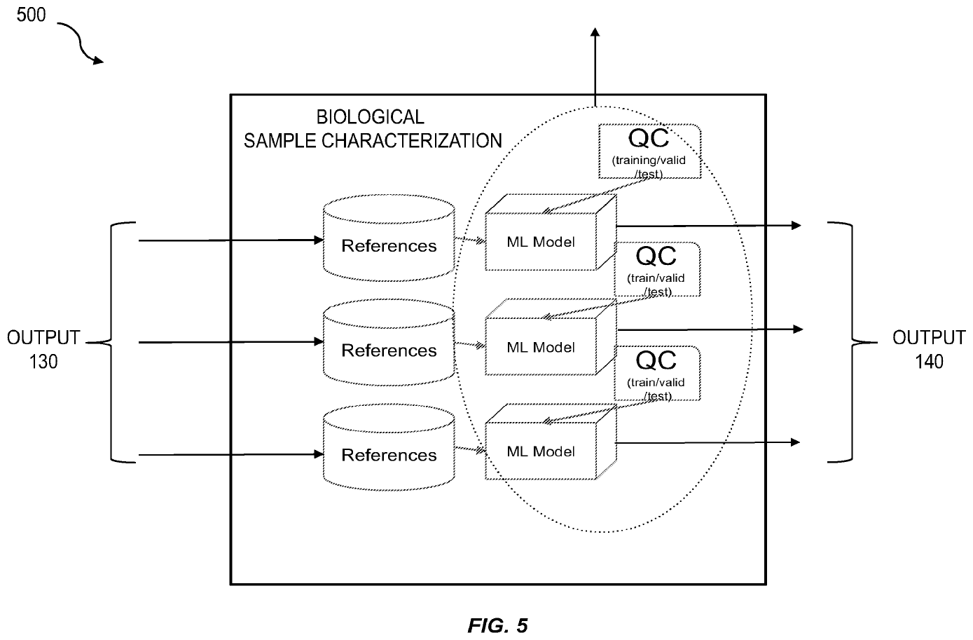

At step 208, the feature set can be provided to an ML model characterized by the selected ML model type. The first ML model configured to characterize the first sample. FIG. 5 illustrates an exemplary method for characterizing biological sample using machine learning algorithm. For example, one of the ML models in biological sample characterization can be selected (e.g., based on user-selected analysis). The selected ML model can receive the output 130 that can include the feature set and the associated metadata. Based on this information the selected ML model can perform generate a final output 140 that can include classification of an analyte, concentration of analyte in the sample, etc.

In some implementations, the method can include selecting a second ML model having the first ML model type, and determining that the second ML model requires further training. The method can further include training, using a training model, the second ML model based on training data including one or more of first sample data, metadata associated with detection of current measurement signal (e.g., provided by the user) and previously generated output of the second ML model (e.g., training data from training data database). The method can also include generating an output by the second ML model configured to receive the feature set and user defined metadata as an input.

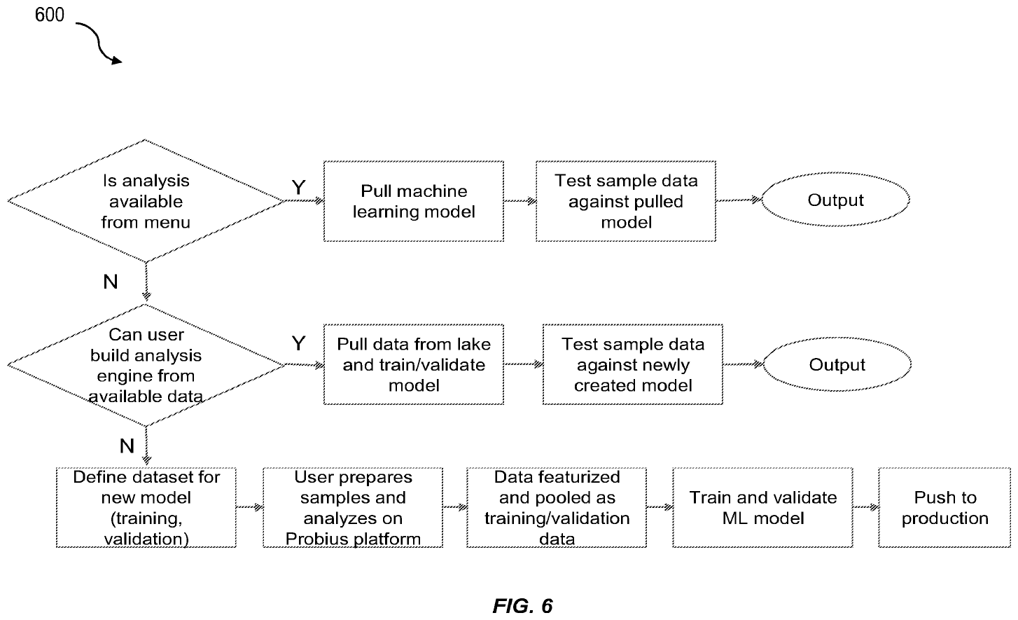

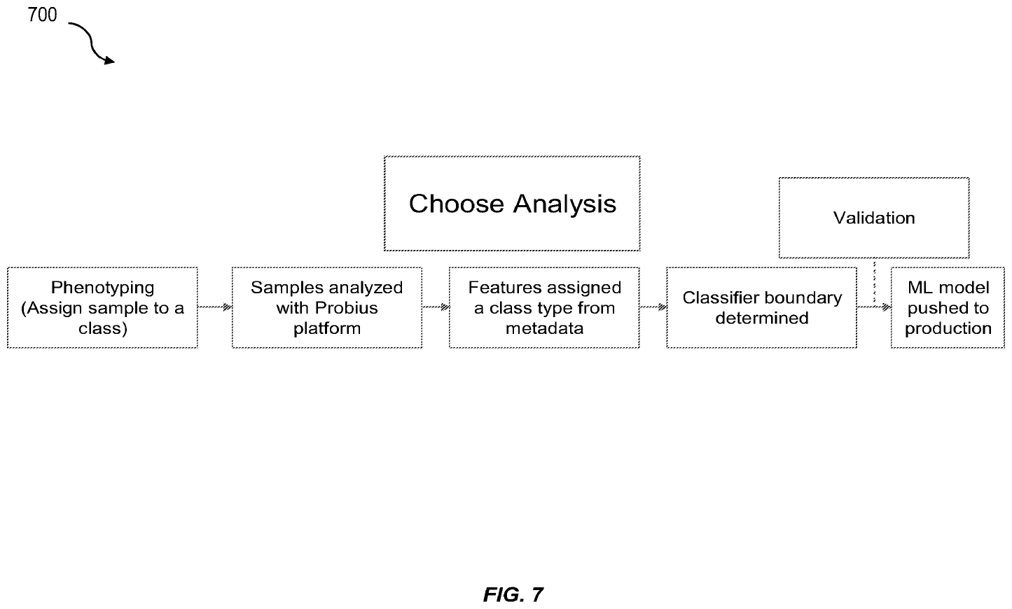

FIG. 6 illustrates an exemplary flow-chart for selecting a machine learning algorithm for the characterization of biological sample. In some implementations, it may be determined if a trained ML (e.g., first ML model) exists. If a trained ML model does not exist, a determination can be made if an existing model (e.g., second ML model) can be trained to perform the desired analysis (e.g., classification, quantification of analytes). If such a model exists, it is selected and trained. If an existing model may not be trained, a determination can be made that a new training dataset can be defined for training and validation and an ML model (e.g., third ML model) associated with the new training dataset can be used to perform the desired analysis.

In some implementation, the second ML model can be trained to assign a class type associated with the first sample (which can be included in the user-defined metadata received at step 202 of FIG. 2). The second ML model can be a classifier configured to assign the class to an analyte in the sample. The training data can be based on one or more samples assigned the class type (e.g., previously analyzed samples using the method described herein), wherein training the classifier includes determining a classifier boundary. The method can further include assigning the class type to the analyte in the first sample using the trained second ML to assign a class to the sample. FIG. 7 illustrates an exemplary flow-chart for classifying a target phenotype in a sample.

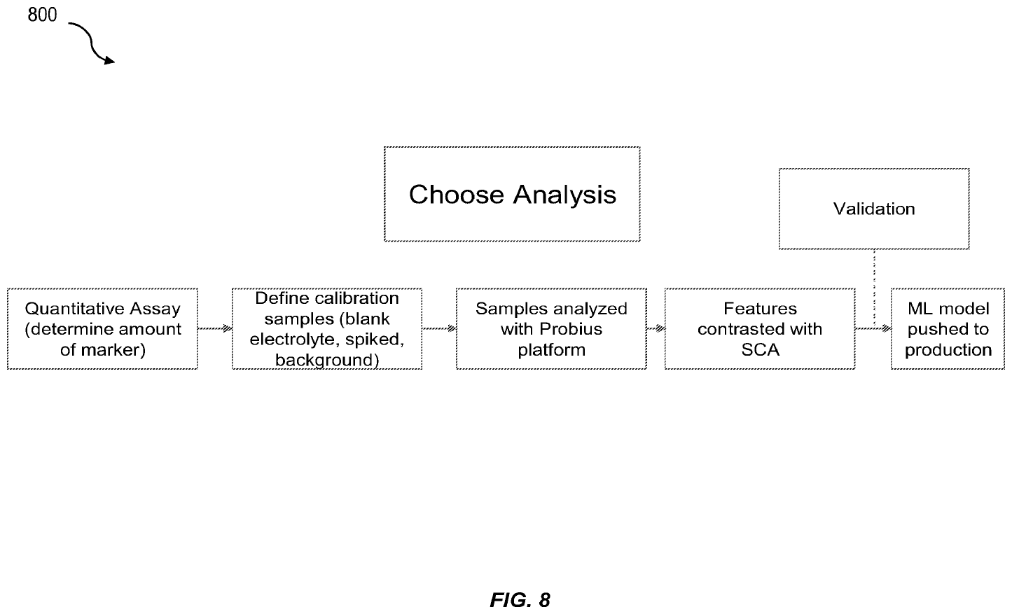

In some implementations, the method can further include defining calibration analyte samples (e.g., redox specie with the biological matrix with nominal levels of the analyte or without the analyte [e.g., glucose], redox specie with various concentration of the analyte, redox specie with varying concentrations of the analyte without the biological matrix, redox specie with varying concentration, redox specie with varying concentrations of chemically and structurally similar analytes without the biological matrix), and analyzing the calibration analyte samples. The method can further include training the second ML algorithm based on a Scattered Component Analysis (SCA) to determine a projection vector that maximizes similarity to analyte-specific reference sample data while minimizing similarity to matrix-specific reference data and/or similarity to chemically and structurally similar analyte reference data, to digitally subtract the contribution of the background and other similar analytes to the signal. The method also includes determining a concentration of the analyte by at least projecting, by the trained second ML algorithm, the sample data onto the projection vector. FIG. 8 illustrates an exemplary flow-chart for quantifying a target analyte in a sample

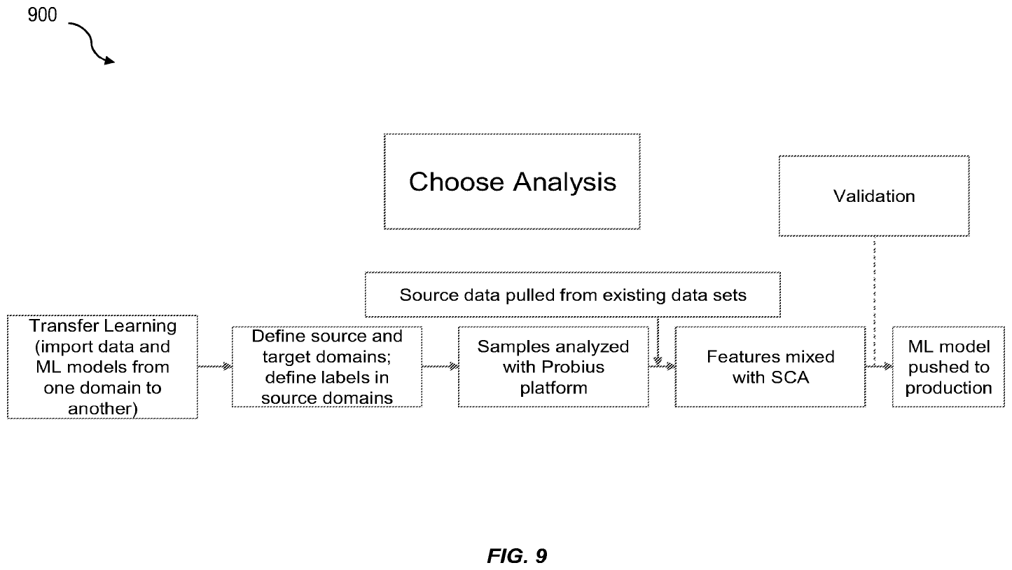

In some implementations, the method can include determining that an ML model having the first ML model type does not exist and identifying a second sample based on a predetermined relationship with the first sample. This is referred to as transferred learning (e.g., an exemplary transferred learning method is described in FIG. 9). The method can further include identifying a third ML model and second training data associated with the second sample. The second training data can include one or more of the second sample data, metadata associated with detection of a current measurement signal associated with the second sample (e.g., provided by the user) and previously generated output of the third ML model (e.g., training data from training data database). The method can further including training, using a training model, the third ML model based on the second training data and generating an output (e.g., classification of an analyte, quantification of analyte in the sample, etc.) by the third ML model configured to receive the feature set and user defined metadata as an input.

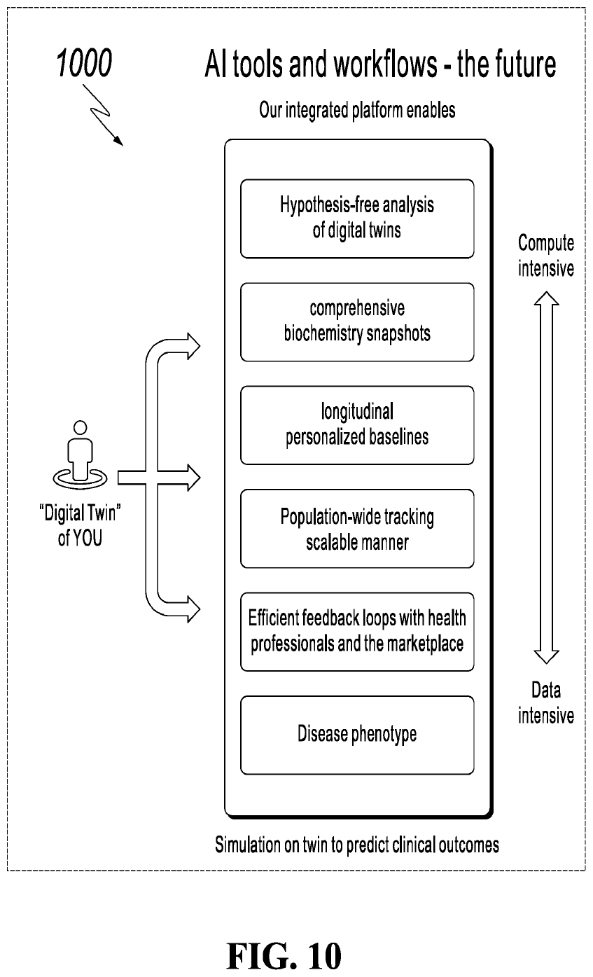

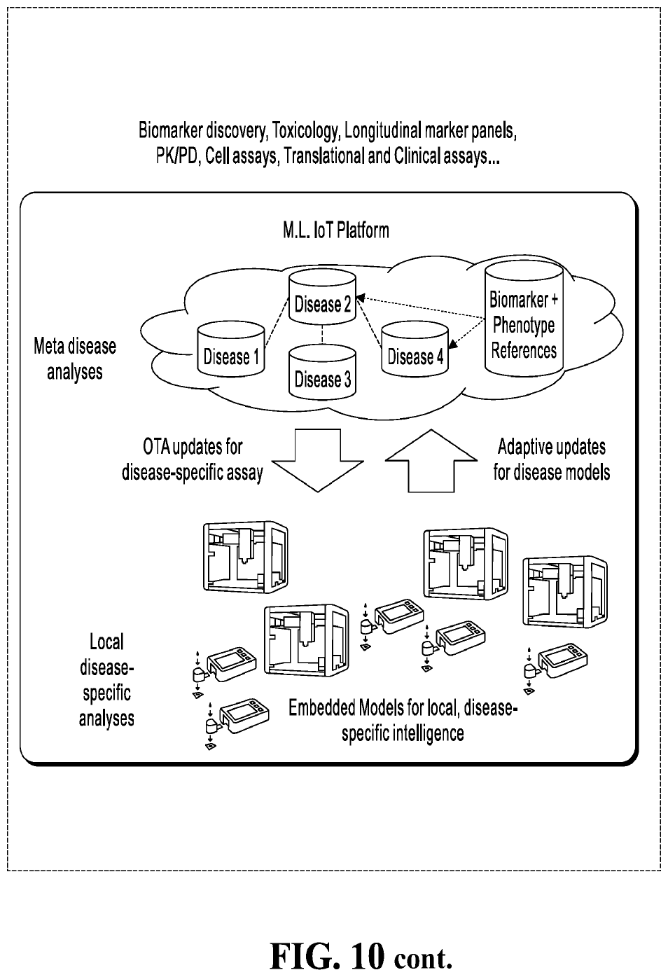

FIG. 10 illustrates and exemplary decentralized deployment of ML (or Artificial intelligence [AI]) driven workflow. From the hypothesis-free generation of the digital twin, diagnoses of disease phenotypes, longitudinal and epidemiological tracking of the phenotypes as well characterization of disease-specific digital features that can be used to triage models and patients can be enabled. The AI-driven workflow can evolve into a decentralized deployment in the future, to a) preserve data security (keep data close to point-of-generation), b) reduce (e.g., minimize) transmission bandwidth consumption and c) enable rapid turnaround of results and as the ML models mature, to leverage the cloud infrastructure (e.g., for discovery applications only).

The local deployment of robust disease models can facilitate quick identification of phenotypes or analytes. The cloud can serve as the primary repository of the disease models, where the training and validation of the models will happen. However, the locally generated data can be leveraged for further training (e.g., when warranted). For example, model for Influenza A, B changes because of yearly mutation of pathogen. The inability of the existing models to accurately predict the disease incidents could trigger the cloud based workflows to provide an over-the-air update to the edge-localized models. Alternatively, a priori knowledge of a new disease phenotype can trigger the over-the-air updates to the local embedded models, without there being a trigger initiated from the edge). This two-way communication between the cloud and edge can enable an adaptive response to biological evolution.

In addition, the cloud can be utilized primarily for discovery, where interdependencies between different disease models would be mapped to specific biomarker profiles. The same toolkit of transfer learning, scatter component analysis etc. with the help of reference/training data can be leveraged to discover these interdependencies which would help triage for specific biochemical signaling pathways and biomarkers for therapy discovery.

### EXAMPLES

Additional embodiments are disclosed in further detail in the following examples, which are provided by way of illustration and are not in any way intended to limit the scope of this disclosure or the claims.

### Example 1

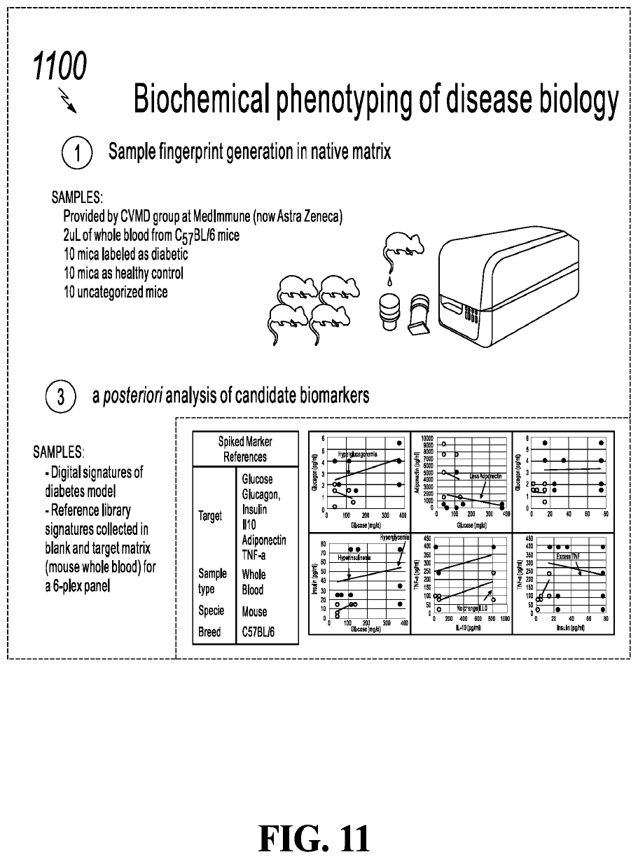

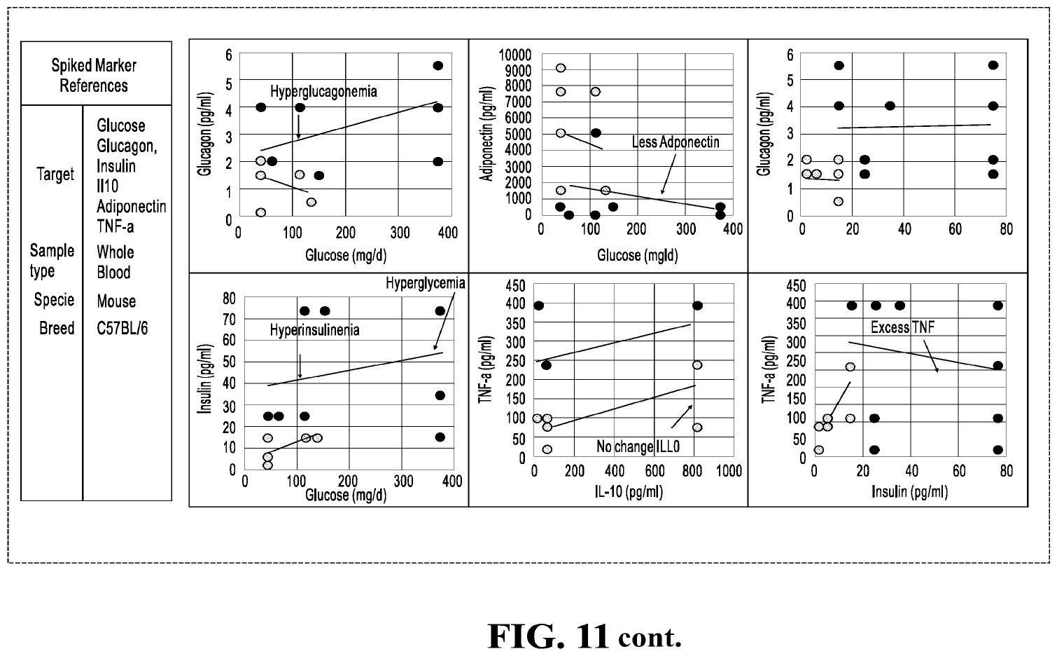

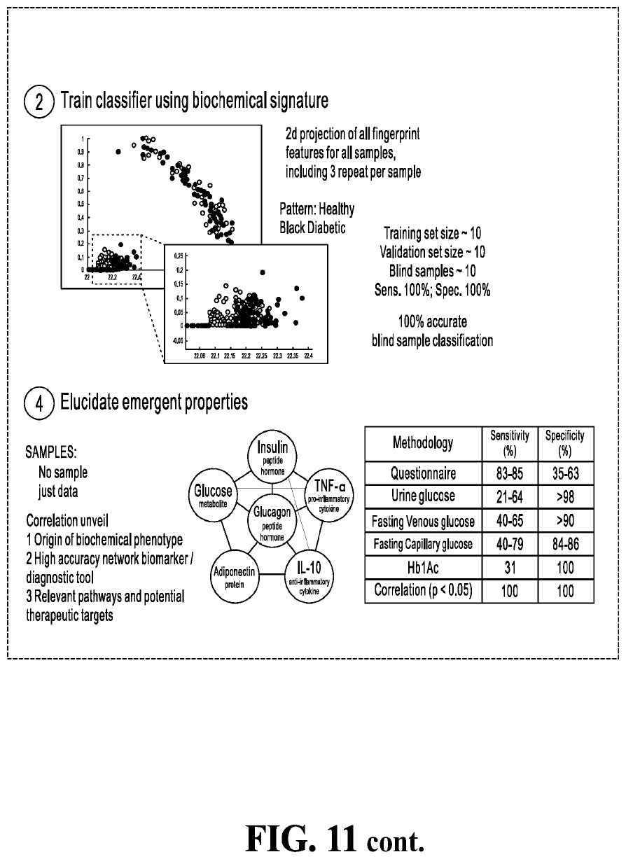

This Example describes a non-limiting exemplary method for biochemical phenotyping of disease biology as illustrated in FIG. 11. In this example, the quantum vibrational signature from the sensor platform is used to triage samples between healthy and diseased samples in a native, unprepared native matrix. In the first step of the method a digital fingerprint of the native matrix (e.g., current measurement data detected by applying a voltage signal across the sample by the sensor platform) is generated. The sample includes 2 microliters of whole blood from C₅₇BL/6 mice. 10 mice are labelled as diabetic, 10 mice are labelled as healthy control and 10 mice are uncategorized. A classifier is trained to identify the diabetic and healthy samples. The training set size is 10, the validation set size is 10, the blind sample size is 10. A 2D projection of all fingerprint features for all samples including 3 repeats per sample is illustrated in FIG. 11. The classifier classified the blind samples with a hundred percent accuracy.

FIG. 11 further illustrates a posteriori analysis of candidate biomarkers that is indicative of disease phenotype. The a posteriori analysis is based on digital signatures of diabetes model, reference library signatures collected in blank and target matrix (mouse whole blood) for a 6-plex panel. Additionally, emergent properties of the candidate biomarker can be identified based on correlations and co-expression of biomarkers. This can allow for re-gaining the accuracy and understanding more about the disease biology and how to effectively treat it. The correlations unveil (1) origin of biochemical phenotype, (2) high accuracy network biomarker/diagnostic tool and (3) relevant pathways and potential therapeutics targets.

### Example 2

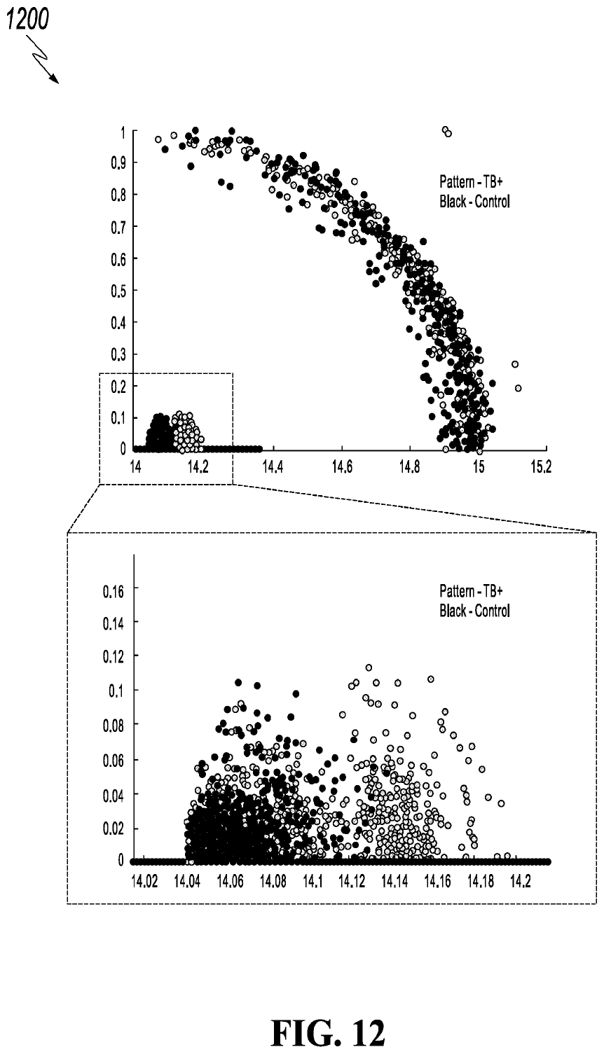

This Example describes a non-limiting exemplary method for phenotyping of tuberculosis in human plasma samples as illustrated in FIG. 12. Each year, 10 million people are infected with tuberculosis with a mortality rate of 1.5 million mortality/year. Tuberculosis is highly infectious (R₀˜ 2.5-4 in crowded environments). Detection of mycobacterium in sputum can be too late to prevent infection. Additionally diagnosis can be costly/time consuming and no fieldable screening solution is available to enable mass testing. In addition, diagnosing TB from plasma samples mitigates the need for biohazard protection protocols for the clinical users of the tool, since the mycobacterium has been removed from the sample.

Eleven samples are used to identify unique TB-specific signature. 20 blind samples are tested for determining performance. Nearest neighbors are grouped using a clustering approach. “Shotgun” feature separation is achieved without appealing to specific markers (suited for TB, where non-pathogenic markers unknown). This example demonstrates diagnostics as a service (DaaS) capability for analysis of human patient samples, where the discerning features of the disease are determined directly from the sample signature, without direct presence of the infecting pathogen. 31 samples from three clinical sites are obtained. ML model predictors of TB phenotypes in plasma using 2 microliter sample aliquots are developed. Small (˜11) population of samples are used to train high fidelity classifier.

### Example 3

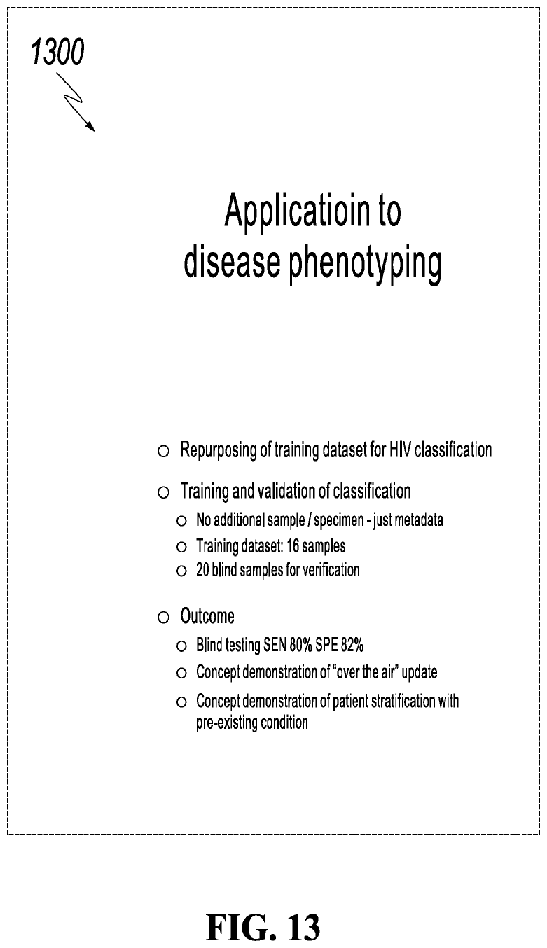

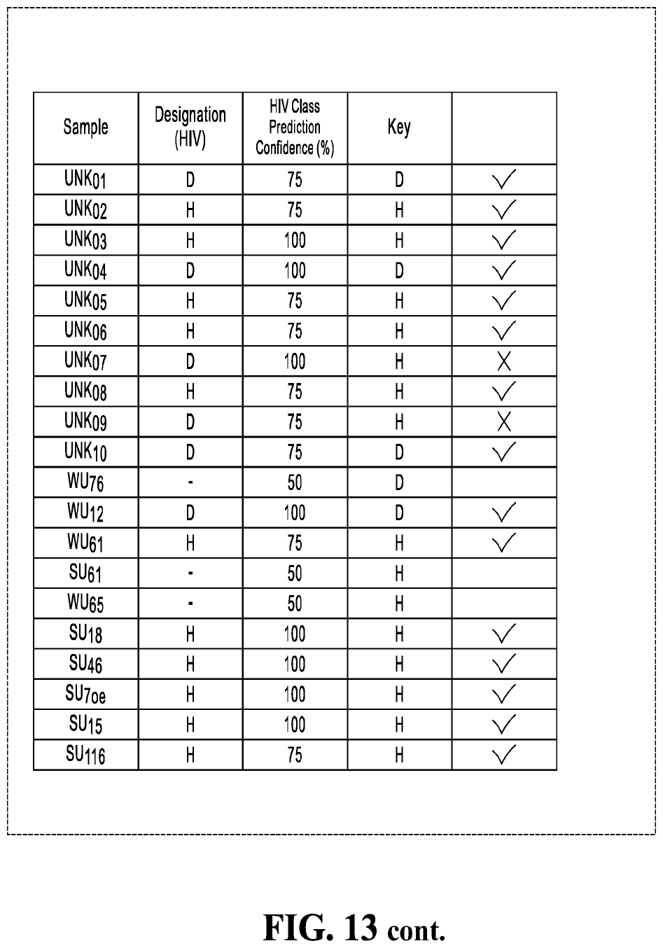

This Example describes a non-limiting exemplary implementation of HIV classification as illustrated in FIG. 13. The training dataset used in example is repurposed for training the classifier for HIV classification. Training data set includes 16 samples and 20 blind samples are verified. No additional sample or specimen data is needed. Additional metadata on existing samples are used to repurpose the classifier developed for the TB application, but to diagnose HIV infection in the same samples.

### Example

This Example describes another exemplary method of basic binding assay. The method includes tracking intensity of individual features within feature-sets in the sample phenotype as function of antigen titer. This can allow for correlation binding between antigen and sample constituents to evolving components of the acquired feature-set (acquired from samples measured on sensor platforms described herein and corresponding to different titers). The workflow described herein could also test concentration of markers before and after introduction of the antigen titer to provide quantitative estimates of the introduced biological perturbation.

### Example 5

This Example describes a non-limiting exemplary method for longitudinal measurement of disease rate, therapy efficacy and toxicity in animal and human models. Time-based snapshots of model biology are captured, where the model is used to understand the expression of a disease, effectiveness of a therapeutic intervention or toxicity of the investigated therapy. The time-evolution of the model phenotype can be tracked either as a function of the evolution of the sample features within acquired feature-sets or as a function of the changing concentrations of specific biomarkers in the sample.

### Example 6

This Example describes a non-limiting exemplary method for tracking longitudinal health of individuals. Sensor platform described herein are leveraged to track a personalized, individualized snapshot of health, where the feature-sets acquired from biological samples extracted non-invasively from a healthy individual (e.g. pin-prick blood draw or urine analysis) at defined time intervals describe a health baseline for that individual, inclusive of diurnal, nocturnal and other time incremental variations. Detection of statistically significant deviations from the baseline could provide early warning signatures of infections and/or chronic pathologies, in terms of the biased components of the sample feature-sets, which could be further translated into changing expressions of different analytes in the sample.

### Example 7

This Example describes a non-limiting exemplary method for epidemiological disease tracking across multiple populations and geographies. Besides capturing disease signatures as high dimensional feature signals in individuals over time, the sensor platform described herein track similar pathologies and associated co-morbidities in populations of patients across different demographics, ethnicities and geographies. The disease tracking functionality is enabled to be conducted longitudinally across groups of individuals, and the tracking could also be conducted to measure disease evolution (as expressed in feature evolution or changing marker levels) in different groups of people.

### Example 8

This Example describes a non-limiting exemplary method for screening with digital phenotype acquisition. The sensor platform described herein enables a mathematical transformation of disease biology into a set of signal feature-sets, which when acquired over a statistically significant population set, can serve as a reference digital signature for the expression of the disease biology for the sample in which the features are measured (blood, plasma, serum, urine etc.). These reference signatures can be leveraged for (a) Screening/triaging patients in diagnostic settings within hospitals or outpatient clinics, potentially at point-of-sampling or point-of-care; (b) screening for most suitable candidates for prospective clinical therapy trials, from a population of available patients; (c) determining the most suitable cellular/animal model for the study of the disease and for testing of therapy candidates in preclinical settings, based on the concurrence of disease features as expressed in animal/cellular model samples versus those expressed in human populations; and (d) providing recommendations for model development for preclinical disease and therapy investigations by defining optimal features required for proper representation of disease pathology in models.

### Example 9

This Example describes a non-limiting exemplary method for a meta-recommendation engine. The sensor platform described herein aggregates the many assays, workflows, disease & therapy studies across research groups and geographies to provide researchers with a tool to collaborate and share their findings where applicable. In addition, based on the insight aggregated from the multiple workflows accessed in the analysis stack, the system provides active recommendations on the directions of future research.

### Example 10

This Example describes a non-limiting exemplary method for inline quality assessment of finished/intermediate products. The sensor platform described herein can be used as a tool to assess the quality of a finished or intermediate product resulting from an industrial production process, where product phenotypic feature-sets can be compared against defined references of ‘acceptable’ products passing quality control. Product samples from batches that pass and fail quality control (assessed using other gold standard approaches like culture) are analyzed with the sensor platform to generate the appropriate reference feature-sets that are metadata tagged as ‘acceptable’ and ‘unacceptable’ respectively, and these reference feature-sets would be leveraged for comparison against a product feature-set to assess quality. Alternatively, the product feature-set can be assayed for intensity of a specific analyte using the sensor platform, which is directly correlated with product spoilage (e.g. Salmonella in packaged lettuce) to assess product quality.

### Example 10

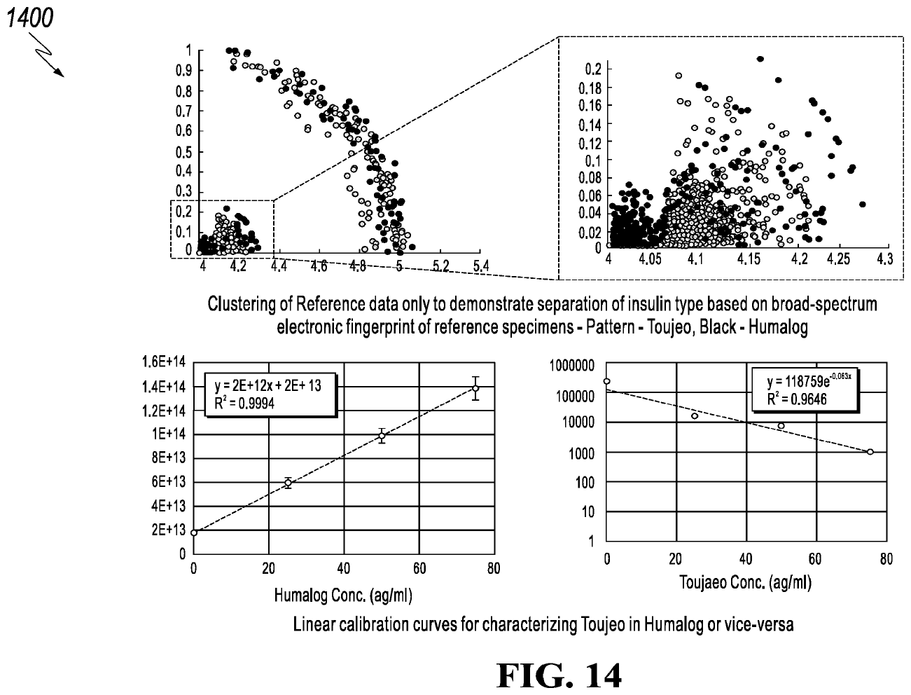

This Example illustrates the characterization of two closely related chemical species in a mixture of the two compounds, where the two similar species have vastly different physiological impacts when ingested as drug compounds (FIG. 14). Insulin Humalog and Toujeo are two isoforms of insulin, where Humalog induces a short acting change in glycemic concentration in the blood, whereas Toujeo induces long-acting regulation of blood glucose. Basic cluster-based phenotyping demonstrates the ability to differentiate one type of insulin isoform from the other for pure samples of each. In addition, for samples comprised of mixtures of the two isoforms, an SCA-based approach is utilized to estimate projection vectors for Humalog and Toujeo-specific signals from the direct measurement of the mixture samples and the pure samples. The normalized projection vector intensity, when plotted against the known concentration of the target analyte, displays a linear dependence, which can be leveraged to calibrate measurements of Humalog and Toujeo in batch mixtures

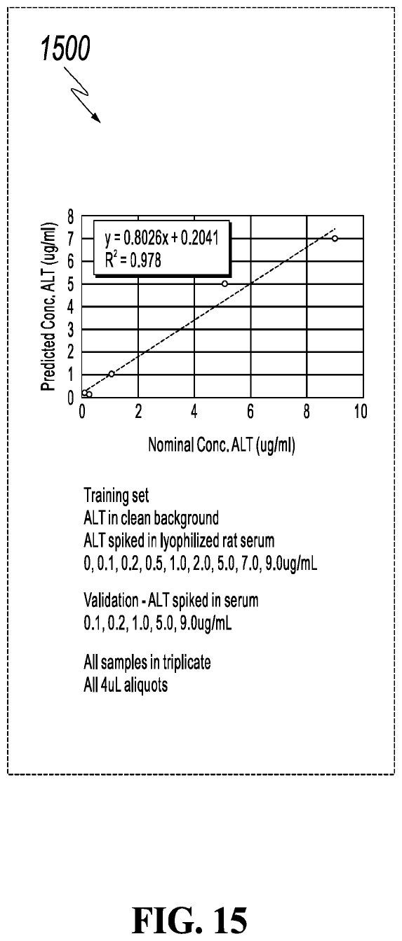

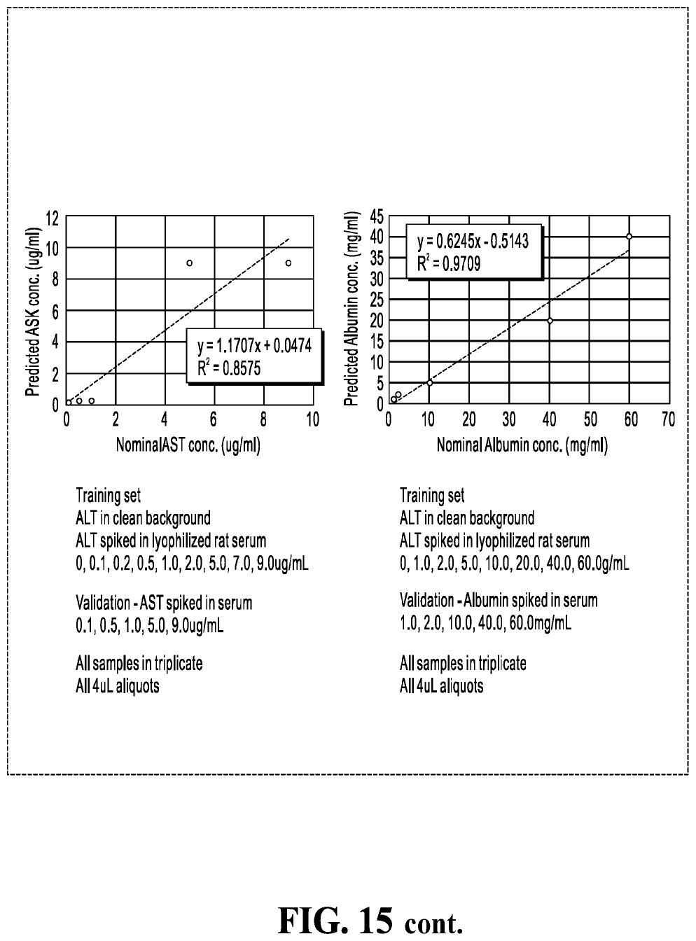

Alternatively or in addition, as illustrated in FIG. 15, further quantitative analysis of three biomarkers in rat serum samples that serve as markers for liver toxicity-liver enzymes ALT, AST and serum Albumin. For each marker, a set of specific training samples is used to develop models to predict the concentration of the marker in rat serum. SCA-based approaches are used to determine analyte-specific projection vectors, to isolate the analyte signal from that of the serum matrix. The as-determined signal projections are used to predict the expression of the markers in a set of validation samples (samples that have not been utilized for prior training).

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. Any recitation herein of the term “comprising”, particularly in a description of components of a composition or in a description of steps of a method, is understood to encompass those compositions and methods consisting essentially of and consisting of the recited components or steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as dis-cussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

1. A method for characterizing biological samples, the method comprising:
(a) receiving data comprising current and voltage measurement data associated with a first sample by at least a sensor platform, metadata associated with the sensor platform, and a user-selected analysis to be performed on the current measurement data, wherein the current measurement data includes current measurement signal data as a function of voltage applied by the sensor platform on the first sample and a measurement time and voltage measurement data includes voltage measurement signal as function of applied set point voltage and a measurement time;
(b) generating a feature set comprising a plurality of coefficients by at least (i) selecting a set of basis functions from a plurality of predetermined learner functions indicative of properties of the electrochemical charge transfer at a sensor interface of the sensor platform, and (ii) generating the plurality of coefficients by at least projecting the current measurement data on the set of basis functions;
(c) selecting a first Machine Learning (ML) model type from a predetermined set of ML model types, the selecting based on the received user-selected analysis; and
(d) providing the feature set to an ML model characterized by the selected ML model type, the first ML model configured to characterize the first sample.

2. The method of claim 1, wherein the metadata associated with the sensor platform includes physical properties of the sensor platform indicative of the electrochemical charge transfer at the sensor interface and/or operational properties of the sensor platform associated with detection of the current measurement signal.

3. The method of claim 1, wherein the received data further includes one or more of (a) data of the source of the first sample, (b) quantitative information associated with analyte species determined from other analysis methods; (c) date and time of first sample collection, storage and re-thaw; (d) one or more quality controls applied to the first sample during collection, storage; (e) any quality control applied to first sample just before analysis; (f) information about co-morbidities of first sample source; (g) disease-relevant phenotype for first sample.

4. The method of claim 1, wherein selecting the set of basis functions includes:
selecting a first set of learner functions and a second set of learner functions from the plurality of predetermined learner functions;
fitting the current measurement signal data with the first set of learner functions and the second set of learner function; and
calculating a first prediction error and a second prediction error associated with the fitting of the current measurement signal with the first set of learner function and the second set of learner function, respectively.

5. The method of claim 4, further comprising selecting one of the first set of learner functions and the second set of learner functions based on the first prediction error and the second prediction error.

6. The method of claim 5, further comprising selecting the first set of learner functions wherein the first prediction error is smaller than the second prediction error.

7. The method of claim 1, further comprising:
selecting a first ML model having the first ML model type, wherein the first trained ML model is characterized by the first model type;
determining that the first ML model does not require further training; and
generating an output by the first ML model configured to receive the feature set and user defined metadata as an input.

8. The method of claim 7, wherein the user specified analysis includes assigning a class to an analyte in the first sample and wherein the first ML model is a classifier configured to assign the class to the analyte.

9. The method of claim 8, wherein the user-specified analysis includes quantification of concentration of an analyte in the first sample.

10. The method of claim 1, further comprising:
selecting a second ML model having the first ML model type, wherein the first trained ML model is characterized by the first model type;
determining that the second ML model requires further training;
training, using a training model, the second ML model based on training data including one or more of first sample data, metadata associated with detection of current measurement signal and previously generated output of the second ML model;
generating an output by the second ML model configured to receive the feature set and user defined metadata as an input.

11. The method of claim 10, further includes:
training the second ML model to assign a class type associated with the first sample, wherein the second ML model is a classifier configured to assign the class to an analyte, wherein the training data is based on one or more samples assigned the class type, wherein training the classifier includes determining classifier boundary; and
assigning the class type to the analyte in the first sample using the trained second ML to assign a class to the sample.

12. The method of claim 10, further includes:
defining calibration analyte samples;
analyzing the calibration analyte samples;
training the second ML algorithm based on a Scattered Component Analysis (SCA) to determine a projection vector that maximizes similarity to analyte-specific reference sample data while minimizing similarity to matrix-specific reference data and/or similarity to chemically and structurally similar analyte reference data, to digitally subtract the contribution of the background and other similar analytes to the signal; and
determining a concentration of the analyte by at least projecting, by the trained second ML algorithm, the sample data onto the projection vector.

13. The method of claim 1, further comprising:
determining that an ML model having the first ML model type does not exist;
identifying a second sample based on a predetermined relationship with the first sample;
identifying a third ML model and second training data associated with the second sample, the second training data including one or more of the second sample data, metadata associated with detection of a current measurement signal associated with the second sample and previously generated output of the third ML model;
training, using a training model, the third ML model based on the second training data; and
generating an output by the third ML model configured to receive the feature set and user defined metadata as an input.

14. A system comprising:
at least one data processor;
memory coupled to the at least one data processor, the memory storing instructions to cause the at least one data processor to perform operations comprising:
(a) receiving data comprising current and voltage measurement data associated with a first sample by at least a sensor platform, metadata associated with the sensor platform, and a user-selected analysis to be performed on the current measurement data, wherein the current measurement data includes current measurement signal data as a function of voltage applied by the sensor platform on the first sample and a measurement time and voltage measurement data includes voltage measurement signal as function of applied set point voltage and a measurement time;
(b) generating a feature set comprising a plurality of coefficients by at least (i) selecting a set of basis functions from a plurality of predetermined learner functions indicative of properties of the electrochemical charge transfer at a sensor interface of the sensor platform, and (ii) generating the plurality of coefficients by at least projecting the current measurement data on the set of basis functions;
(c) selecting a first Machine Learning (ML) model type from a predetermined set of ML model types, the selecting based on the received user-selected analysis; and
(d) providing the feature set to an ML model characterized by the selected ML model type, the first ML model configured to characterize the first sample.

15. The system of claim 14, wherein the metadata associated with the sensor platform includes physical properties of the sensor platform indicative of the electrochemical charge transfer at the sensor interface and/or operational properties of the sensor platform associated with detection of the current measurement signal.

16. The system of claim 14, wherein the received data further includes one or more of (a) data of the source of the first sample, (b) quantitative information associated with analyte species determined from other analysis methods; (c) date and time of first sample collection, storage and re-thaw; (d) one or more quality controls applied to the first sample during collection, storage; (e) any quality control applied to first sample just before analysis; (f) information about co-morbidities of first sample source; (g) disease-relevant phenotype for first sample.

17. The system of claim 14, wherein selecting the set of basis functions includes:
selecting a first set of learner functions and a second set of learner functions from the plurality of predetermined learner functions;
fitting the current measurement signal data with the first set of learner functions and the second set of learner function; and
calculating a first prediction error and a second prediction error associated with the fitting of the current measurement signal with the first set of learner function and the second set of learner function, respectively.

18. The system of claim 17, wherein the operations further comprising selecting one of the first set of learner functions and the second set of learner functions based on the first prediction error and the second prediction error.

19. The system of claim 18, wherein the operations further comprising selecting the first set of learner functions wherein the first prediction error is smaller than the second prediction error.

20. The system of claim 14, wherein the operations further comprising:
selecting a first ML model having the first ML model type, wherein the first trained ML model is characterized by the first model type;
determining that the first ML model does not require further training; and
generating an output by the first ML model configured to receive the feature set and user defined metadata as an input.

21. The system of claim 20, wherein the user specified analysis includes assigning a class to an analyte in the first sample and wherein the first ML model is a classifier configured to assign the class to the analyte.

22. The system of claim 21, wherein the user-specified analysis includes quantification of concentration of an analyte in the first sample.

23. The system of claim 14, wherein the operations further comprising:
selecting a second ML model having the first ML model type, wherein the first trained ML model is characterized by the first model type;
determining that the second ML model requires further training;
training, using a training model, the second ML model based on training data including one or more of first sample data, metadata associated with detection of current measurement signal and previously generated output of the second ML model;
generating an output by the second ML model configured to receive the feature set and user defined metadata as an input.

24. The system of claim 21, wherein the operations further include:
training the second ML model to assign a class type associated with the first sample, wherein the second ML model is a classifier configured to assign the class to an analyte, wherein the training data is based on one or more samples assigned the class type, wherein training the classifier includes determining classifier boundary; and
assigning the class type to the analyte in the first sample using the trained second ML to assign a class to the sample.

25. The system of claim 21, wherein the operations further include:
defining calibration analyte samples;
analyzing the calibration analyte samples;
training the second ML algorithm based on a Scattered Component Analysis (SCA) to determine a projection vector that maximizes similarity to analyte-specific reference sample data while minimizing similarity to matrix-specific reference data and/or similarity to chemically and structurally similar analyte reference data, to digitally subtract the contribution of the background and other similar analytes to the signal; and
determining a concentration of the analyte by at least projecting, by the trained second ML algorithm, the sample data onto the projection vector.

26. The system of claim 14, wherein the operations further comprising:
determining that an ML model having the first ML model type does not exist;
identifying a second sample based on a predetermined relationship with the first sample;
identifying a third ML model and second training data associated with the second sample, the second training data including one or more of the second sample data, metadata associated with detection of a current measurement signal associated with the second sample and previously generated output of the third ML model;
training, using a training model, the third ML model based on the second training data; and
generating an output by the third ML model configured to receive the feature set and user defined metadata as an input.

27. A computer program product comprising a non-transitory machine-readable medium storing instructions that, when executed by at least one programmable processor that comprises at least one physical core and a plurality of logical cores, cause the at least one programmable processor to perform operations comprising:
(a) receiving data comprising current and voltage measurement data associated with a first sample by at least a sensor platform, metadata associated with the sensor platform, and a user-selected analysis to be performed on the current measurement data, wherein the current measurement data includes current measurement signal data as a function of voltage applied by the sensor platform on the first sample and a measurement time and voltage measurement data includes voltage measurement signal as function of applied set point voltage and a measurement time;
(b) generating a feature set comprising a plurality of coefficients by at least (i) selecting a set of basis functions from a plurality of predetermined learner functions indicative of properties of the electrochemical charge transfer at a sensor interface of the sensor platform, and (ii) generating the plurality of coefficients by at least projecting the current measurement data on the set of basis functions;
(c) selecting a first Machine Learning (ML) model type from a predetermined set of ML model types, the selecting based on the received user-selected analysis; and
(d) providing the feature set to an ML model characterized by the selected ML model type, the first ML model configured to characterize the first sample.
